(19) United States
(12) Patent Application Publication
(10) Pub. No.: US 2017/0023540 A1
(43) Pub. Date: Jan. 26, 2017

(54) METHOD FOR MEASURING THE TRAPPED GAS SATURATION IN A ROCK SAMPLE

(71) Applicant: ENI S.p.A., Roma (IT)

(72) Inventor: Nicola Giovanni BONA, Milano (IT)

(73) Assignee: ENI S.p.A., Roma (IT)

(21) Appl. No.: 15/039,144

(22) PCT Filed: Nov. 27, 2014
(86) PCT No.: PCT/IB2014/066391

§ 371 Date: May 25, 2016

(30) Foreign Application Priority Data
Nov. 28, 2013 (IT) MI2013A001986

(51) Int. Cl. G01N 33/24; G01N 23/04; G01N 24/08

(52) U.S. Cl. CPC G01N 33/241; G01N 24/081; G01N 23/046; G01N 2223/616

(57) ABSTRACT

The present invention concerns a method (100) for measuring the trapped gas saturation in a rock sample, comprising the steps: saturating (101) the porous volume of the rock sample with water; effecting a first imaging phase (102) capable of dividing the sample into a plurality of subsamples and measuring the porosity of each subsample; subjecting the sample of water saturated rock to centrifugation in air (103); effecting a second imaging phase (104) so as to obtain an initial gas saturation map Sgi of the sample; subjecting the rock sample to centrifugation under water (105); effecting a third imaging phase (106) so as to obtain a gas saturation map Sg of the sample; calculating (107) a plurality of Bond number values; associating (108), for each subsample, the gas saturation Sg with the initial gas saturation Sgi and with the Bond number values calculated; identifying (109) the Bond number value beyond which the gas saturation Sg begins to decrease, the value identified representing the critical Bond number; selecting (110) the initial gas saturation values Sgi and gas saturation Sg corresponding to Bond number values lower than the value of said critical Bond number, the gas saturation values Sg selected representing corresponding trapped gas saturation values Sgr.

The present invention refers to a method for measuring the trapped gas saturation in a rock sample that is particularly, but not exclusively, useful in the field of extracting hydrocarbons.

It is known that when gas, for example a natural gas, is extracted from a gas field below which there is an aquifer in hydraulic communication with the gas field itself, the water of such an aquifer is set in motion upwards by the pressure gradient that is generated in the gas field due to the extraction of gas. Such a phenomenon is also called water drive.

Following the water drive phenomenon, part of the gas present in the gas field is pushed and trapped inside the rock formation of the gas field.

By the term “gas saturation” we mean the fraction of the porous volume of a rock that is occupied by gas.

The trapped gas saturation is the fraction of porous volume of rock that, in an area of the gas field that is flooded by the aquifer, contains gas that can no longer be produced because it is trapped and isolated by the water.

The measurement of the trapped gas saturation, therefore, is indicative of the loss of gas that can be extracted consequent to the water drive phenomenon.

Currently, the trapped gas saturation is estimated by measuring such a value in core rock samples.

Four methods are essentially known for measuring trapped gas saturation in a rock sample, respectively called Unsteady-state water injection, Steady-state water injection, Centrifugation under water and Countercurrent imbibition.

In particular, the first three methods belong to the very wide group of the methods for measuring relative permeability curves.

The method called Unsteady-state water injection foresees injecting water, by means of a pump, at a face of a rock sample which is partially saturated with water and gas. Following the injection of water, part of the gas contained in the sample comes out and part of it is trapped inside the pores of the sample itself; by measuring the weight of the sample at the end of the water injection process it is possible to obtain the gas saturation of the examined sample. The Unsteady-state water injection method suffers from some drawbacks.

Indeed, the trapped gas saturation value Sgr obtained depends upon the volume of water that is injected in the analysed rock sample. More in particular, by injecting a small volume of water there is the risk of overestimating the trapped gas saturation Sgr since not all the mobile gas is actually extracted during the experiment; conversely, by injecting a great volume of water there is the risk of underestimating the trapped gas saturation Sgr since part of the non-mobile gas is dissolved by the water and is erroneously extracted.

Indeed, the pressure applied by the pump to the injected water alters the balance between the water and the gas inside the sample promoting the diffusion of gas particles in the flow of water that comes out from the sample. The correct volume of water to be injected represents a compromise between the two opposite trends, but it is not known before beginning the experiment, and it cannot be estimated at a later moment during the quality control step of the results.

The Steady-state water injection method is based upon the simultaneous injection of water and gas inside the rock sample. The injection continues constant until a stationary condition is reached in which the flows of gas and water in outlet are in constant ratio with one another. At this stage the flow rate of gas in inlet is gradually decreased and the gas saturation is gradually measured in stationary conditions or almost-stationary conditions. The Steady-state water injection method, however, does not reproduce the development of the flow of fluids that is characteristic of the gas field and, therefore, the results of such a method cannot all represent the real situation of the gas field.

The Centrifugation under water method consists of centrifuging a cell containing water and a rock sample that is partially saturated with water and partially with gas. The gas saturation of the sample before centrifugation under water is the so-called initial gas saturation Sgi. After centrifuging, the water contained in the cell penetrates inside the rock sample pushing part of the gas out from the sample itself.

By either measuring the weight of the sample at the end of the centrifugation or monitoring the amount of expelled gas while centrifuging, it is possible to obtain the average trapped gas saturation of the sample that has been examined.

The Centrifugation under water method, however, can give inaccurate trapped gas saturation Sgr results and, therefore, results that are not representative of the gas field if the experiment is carried out with a rotation velocity of the centrifuge that is too fast.

Indeed, in such a condition even the gas that would remain trapped in the rock in the gas field tends to come out from the sample for the exceedingly strong centrifuge force that is applied.

The maximum rotation velocity that can be applied is determined by the so-called Bond number Nb, which is an adimensional amount that measures the relationship between the force of gravity that is induced by the centrifuge and the capillary force; such a Bond number Nb must not exceed a certain critical value in order to obtain accurate measurements of gas saturation.

The Bond number is defined as Δρgk/σ (where Δp is the difference in density between water and gas, g is the centrifuge acceleration, k is the permeability and σ is the water-gas interfacial tension) and varies inside the sample as a function of the distance from the rotation axis of the centrifuge and the rotational speed of the centrifuge. The trapped gas saturation Sgr measured with the Centrifugation under water method, therefore, depends both on the Bond number Nb and on the initial gas saturation value Sgi.

Since both the Bond number Nb and the initial gas saturation value Sgi vary in the sample from one point to the next, also the trapped gas saturation Sgr is distributed in a non-homogeneous manner. The trapped gas saturation measured by means of the centrifugation under water, therefore, represents an average saturation value of the sample.

However, in order to characterize the trapped gas saturation due to water drive it is necessary to associate the corresponding Sgr value with a precise Sgi value. Therefore, the use of average values measured on a rock sample which, like what happens with the aforementioned under water centrifugation method can have a very heterogeneous gas saturation distribution, does not ensure accurate results.

The Centrifugation under water method, therefore, is capable of estimating the average saturation of the gas in the rock sample; by processing the data it is then possible to estimate the gas saturation that exists on one of the faces of the sample. For such a face it is possible to determine the Bond number so as to obtain the tern of values Sgi-Sgr-Bond number necessary for characterising the trapped gas saturation. The result obtained, however, represents very partial information, due to the fact that it is limited to only one face of the sample. Moreover, since the critical Bond number value is not known, it is not possible to establish whether the pair of values Sgr−Sgi calculated in relation to one face of the sample actually represents the real situation inside the gas field or not.

The fourth countercurrent imbibition method represents the main and most used method for measuring the trapped gas saturation from water drive.

According to the countercurrent imbibition method, toluene is introduced by imbibition in a rock sample until it is saturated; part of the toluene introduced is then evaporated in air until an average predetermined saturation has been achieved, which represents the initial gas saturation or Sgi. Finally, the rock sample is immersed in a bath of toluene which by imbibition penetrates inside the sample itself and the increase in weight of the sample is measured over time. From the weight of the sample at the end of the imbibition process it is possible to obtain the value of trapped gas saturation Sgr. The value of trapped gas saturation Sgr thus measured is associated with the initial gas saturation Sgi. The process described above is replicated a certain number of times, preferably at least four times, changing the evaporation time so as to obtain at the end four pairs of trapped gas saturation values Sgr as a function of the initial gas saturation Sgi.

The countercurrent imbibition method suffers from some drawbacks.

A first drawback is that the results of such a method can be trapped gas saturation values Sgr that are too high due to the fact that in a countercurrent flow the probability of isolating the non-wetting phase (gas) is greater than with an equi-current flow.

Another drawback of countercurrent imbibition lies in the fact that the initial gas saturation Sgi that is created in the rock sample at the end of the toluene evaporation phase may not be distributed homogeneously in the sample itself. Inside the sample there can indeed be a toluene content that is greater than near to the surfaces of the sample, which are more affected by the evaporation. This can cause errors in the subsequent estimation of the trapped gas saturation Sgr, which requires a homogeneous Sgi.

The purpose of the present invention is that of avoiding the drawbacks mentioned above and in particular that of conceiving a method for measuring the trapped gas saturation in a rock sample that is capable of obtaining more accurate measurements with respect to the known methods.

These and other purposes according to the present invention are achieved by creating a method for measuring the trapped gas saturation in a rock sample as outlined in claim 1.

Further characteristics of a method for measuring the trapped gas saturation in a rock sample are object of the dependent claims.

The characteristics and the advantages of a method for measuring the trapped gas saturation in a rock sample according to the present invention shall become clearer from the following description, given as an example and not for limiting purposes, with reference to the attached schematic drawings, in which:

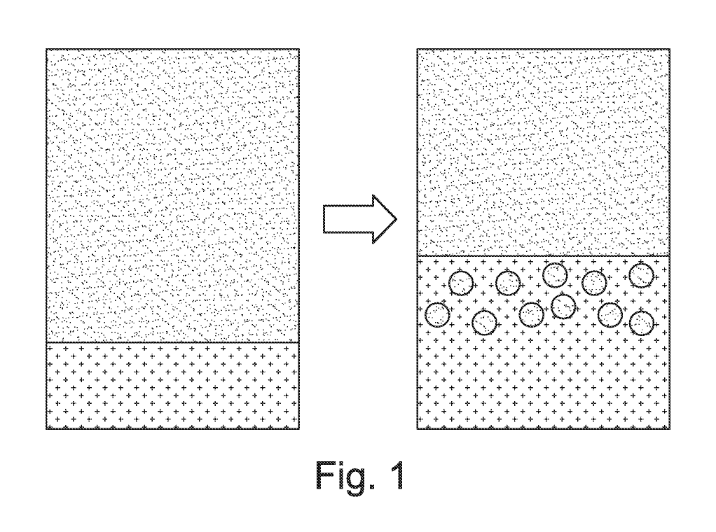

FIG. 1 is a schematic view representing the phenomenon of gas being trapped inside the rock of the gas field following the water drive phenomenon in which the dotted region is occupied by gas and the region with crosses is occupied by water;

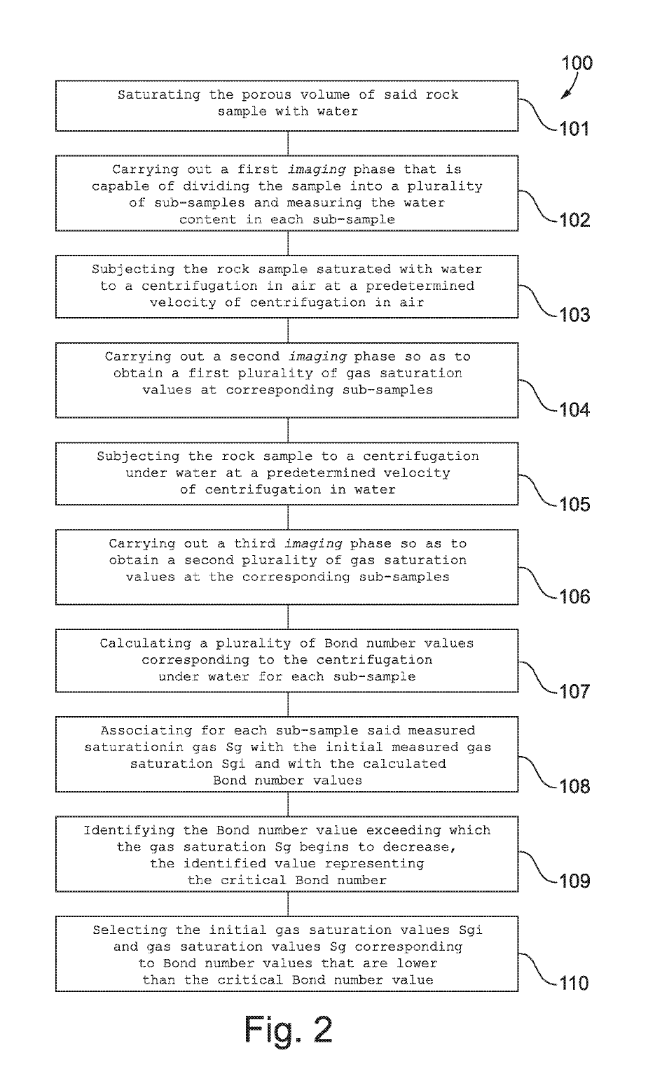

FIG. 2 is a flow chart representing a method for measuring the trapped gas saturation in a rock sample according to the present invention;

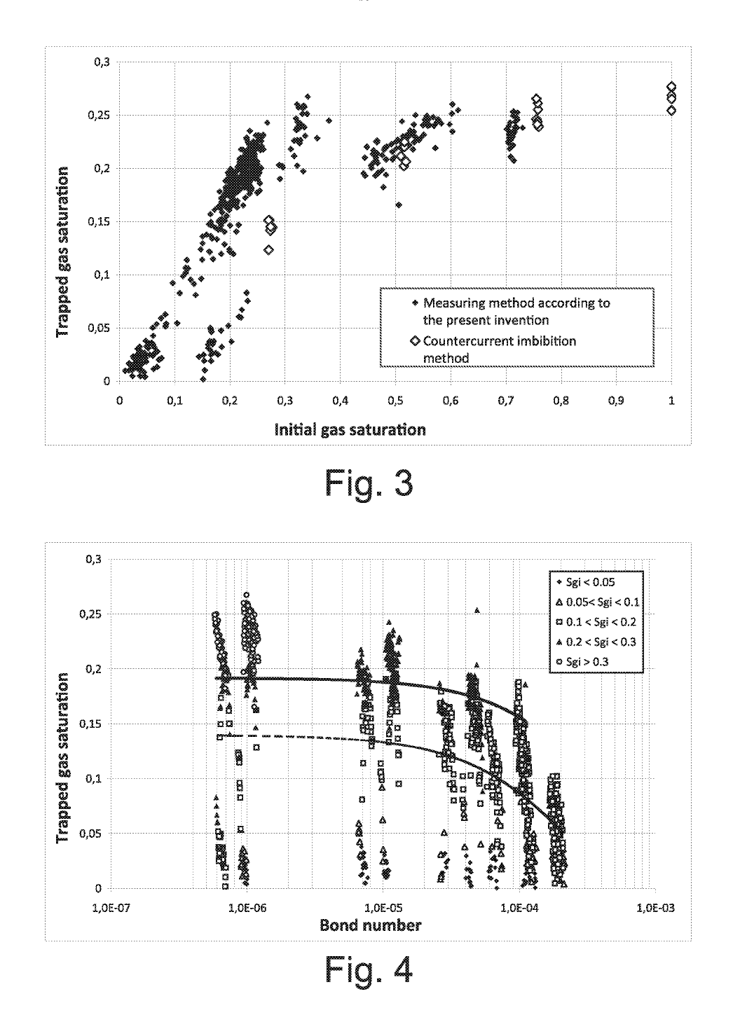

FIG. 3 is a graph showing the trapped gas saturation values as a function of the initial gas saturation values, measured in a rock sample with the method according to the present invention and with the countercurrent imbibition method;

FIG. 4 is a graph, obtained by the method according to the present invention, which shows the trapped gas saturation values as a function of the Bond number and of a plurality of ranges of values of the initial gas saturation;

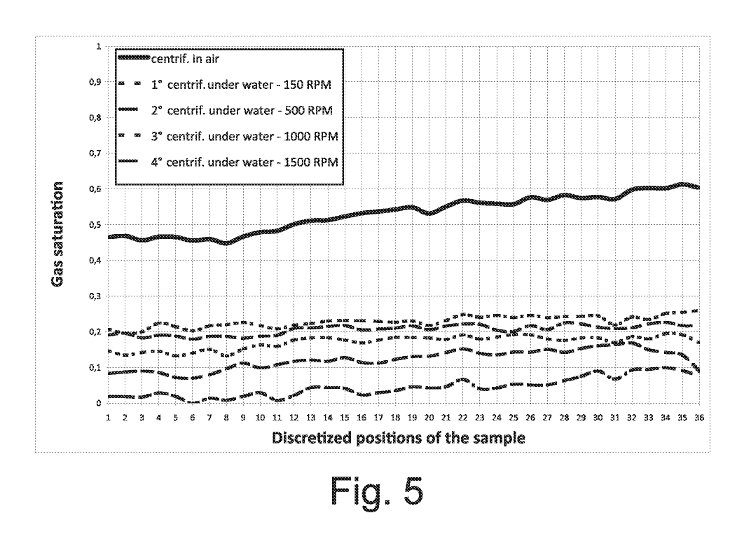

FIG. 5 is a graph, obtained with the method according to the present invention, comprising a plurality of curves corresponding to different values of rotation velocity of the centrifuge and representing the variation of the trapped gas saturation values as a function of the position at which the measurement has been carried out.

With reference to the figures, these show a method for measuring the trapped gas saturation in a rock sample, wholly indicated with reference numeral 100. Such a measuring method 100 comprises the step in which the porous volume of the rock sample is saturated 101 with water. Such a step is carried out by introducing water, for example, through imbibition in the rock sample so as to occupy substantially all of the porous volume of the rock sample.

Subsequently, a first imaging phase 102 is carried out so as to obtain a porosity map of the sample.

In the present description by imaging phase we mean carrying out a method that is capable of dividing the sample into a plurality of subsamples and measuring the water content in each subsample. The water content is the volume of water contained in the sample and it corresponds to the product of the porosity of the rock times its saturation in water times the volume of the sample. The volume of the sample is known beforehand.

When the imaging is carried out on a subsample that is entirely saturated with water, the saturation in water is equal to 1 and from the measured water content it is possible to obtain the porosity of the subsample, by dividing the water content by the volume of the subsample. When the imaging is carried out on a subsample, the pores of which are only partially saturated with water, the water content measured in such a saturation condition divided by the porosity of the subsample and by its volume corresponds to the saturation in water of the subsample.

The gas saturation, therefore, can be obtained from the difference between the maximum saturation, or rather 1, and the measurement of the saturation in water, since the fraction of porous volume which is not occupied by water is occupied by gas.

In the case in which monodimensional imaging is used, the rock sample is discretized along a predetermined direction at a plurality of positions and the result of such a method is a monodimensional map, or rather a curve that relates the water content to the relative position.

On the other hand, in the case in which three-dimensional imaging is used, the rock sample is discretized in a plurality of three-dimensional subsamples and the result is a three-dimensional map that puts the water content and the relative three-dimensional subsample in relation with one another.

The discretization used by the imaging method is preferably such as to divide the sample into a plurality of subsamples that are sufficiently small so as to be able to consider the gas saturation as homogeneously distributed inside them.

Preferably, the imaging phases are effected by means of a monodimensional or three-dimensional tomographic analysis.

Preferably, the imaging phases are effected by means of nuclear magnetic resonance (NMR).

Preferably, the imaging phases are effected by means of Gamma ray analysis.

Preferably, the imaging phases are effected by means of X-ray analysis.

Following the first imaging phase 102, the sample of water saturated rock is subjected to a centrifugation 103 in air at a pre-determined centrifugation velocity.

In such a way part of the water contained inside the sample comes out and the porous volume that is left free by the water is occupied by air, or rather by a gas. Such a porous volume occupied by gas at the end of the centrifugation in air represents the initial gas saturation Sgi.

Therefore, the predetermined velocity of the centrifugation in air is set on the basis of the initial gas saturation with respect to which the trapped gas saturation is desired to be known.

The centrifugation in air is carried out by making a cell full of air containing the sample saturated with water centrifuge about a rotation axis.

For example, in the case in which the distance between the rotation axis and the face of the sample furthest from such an axis is around 25 cm, the centrifugation in air can be carried out with a rotation velocity of around 2000-3000 revs/minute.

After the centrifugation in air 103, the measuring method foresees a second imaging phase 104 so as to obtain a first plurality of gas saturation values at the corresponding subsamples, which obtain an initial gas saturation map Sgi of the sample.

Subsequently, the rock sample is subjected to a centrifugation under water 105 at a pre-determined centrifugation velocity under water.

Conversely to the centrifugation in air, centrifugation under water 105 is carried out by making a cell that is partially full of water containing the sample to centrifuge around a rotation axis.

In such a way the water drive phenomenon is simulated since the water, by effect of the centrifugal force, penetrates inside the sample pushing part of the gas outside the sample itself and promoting the trapping of the remaining part of gas inside the pores of the sample.

The rock sample is fixed onto the base of the cell.

Alternatively, the cell preferably comprises a spacer, arranged between the base of the cell and the sample, configured for keeping the rock sample itself at a predetermined distance from the bottom of said cell.

In order to increase the range of saturation values and the Bond numbers, the sample is arranged as close as possible to the centre of rotation of the centrifuge.

In any case, at the end of the centrifugation under water 105, the sample is subjected to a third imaging phase 106 so as to obtain a second plurality of gas saturation values at the corresponding subsamples, which obtain a gas saturation map Sg of the sample.

Advantageously, the method object of the present invention comprises the step in which a plurality of Bond number values is calculated 107 corresponding to the centrifugation under water for each subsample. So for each subsample the measured gas saturation Sg is associated 108 with the initial gas saturation Sgi, which is measured after the centrifugation in air and with the calculated Bond numbers, generating a tern Sgi-Sg-Nb for each subsample.

The gas saturation values obtained can be considered trapped gas saturation values Sgr which are representative of what occurs in gas fields only if the

Bond number does not exceed a certain critical value Nbc, which based upon the literature is in the order of 10-5, but can be different each time.

As it is possible to observe in FIG. 4, the saturations in gas, at a plurality of ranges of initial gas saturation values Sgi, have values that are substantially constant up to a certain Bond number value Nb and then they start to decrease.

According to the present invention, advantageously the Bond number value beyond which the gas saturation Sg begins to decrease is identified 109; the identified value indeed represents the critical Bond number Nbc.

Once the critical Bond number Nbc has been identified, the initial gas saturation values Sgi and gas saturation values Sg are selected 110, which correspond to the Bond number values that are lower than the critical Bond number value Nbc. The gas saturation values Sg thus selected represent the trapped gas saturation values Sgr.

Such selected values represent a plurality of pairs of values Sgr−Sgi that are representative of the gas field.

Preferably, the centrifugation under water phase 105, the following third imaging phase 106, the phase for calculating the Bond numbers relative to the centrifugation under water 107 and the association phase 108 are repeated for a predetermined number of times M in succession with one another with increasing velocity. These centrifugations with gradually increased velocity progressively decrease the gas saturation inside the rock sample being analysed.

In such a way, the association phases 108 generate a plurality of new terns Sg(k)−Sg(k+1)−Nb(k+1) where Sg(k) represents the gas saturation at the end of k-th centrifugation under water, Sg(k+1) the gas saturation at the end of the (k+1)-th centrifugation under water and Nb (k+1) is the Bond number that is associated with the (k+1)-th centrifugation under water 108.

The plurality of terns Sg(k)−Sg(k+1)−Nb(k+1) obtained with the M centrifugations under water following the first centrifugation is added to the data provided in the graph that is illustrated in FIG. 4 and contributes towards determining the critical Bond number value Nbc 109.

In the case in which a monodimensional imaging method is adopted, the length of the sample is discretized in a number N of positions. With a number of centrifugation under water phases equal to M, M×N independent values of gas saturation Sg are obtained, to which N values measured after the centrifugation in air, are added.

In the particular case of FIG. 3, the data shown was obtained by discretizing the sample in N=36 positions and by making M=5 centrifugation under water phases (rotation velocity: 150, 500, 1000, 1500 and 2000 rpm): this experimental setup provided 36×6=215 gas saturation values. The points shown in FIG. 3 are those whose associated Bond number is lower than the critical value and represent the final output of the measurement.

With the purpose of selecting the trapped gas saturation values Sgr that are representative of the gas field, the gas saturations Sg obtained for each subsample and at the end of each centrifugation under water phase can be shown graphically as a function of the corresponding Bond number (defined by Nb=Δρgk/σ, where Δρ=difference of water-air density indicated in kg/m³; g=centrifugal acceleration in the position considered indicated in m/s²; k=permeability of the sample indicated in m²; σ=interfacial tension indicated in N/m), as for example illustrated in FIG. 4 in which the saturation measurements are effected by means of a monodimensional imaging method.

Once the trapped gas saturation values Sgr which are representative of the behaviour of the gas field have been identified, i.e. those obtained with the velocity of the centrifuge rotation and in positions in the sample in which the Bond number is lower than the critical value that was previously identified, at the position of each subsample two consecutive centrifugation under water phases provide a pair of values Sgi-Sgr. The gas saturation immediately before centrifuging under water represents the value of Sgi and that immediately after is the Sgr value that is associated with it.

From the description made the characteristics of the measuring method object of the present invention should be clear, just as the relative advantages should also be clear.

The combination of the centrifugation under water of the rock sample and of the imaging methods makes it possible to analyse the sample as a set of many sub-regions or sub-samples which can be characterised individually in terms of saturation. Each subsample provides data that is independent from the other subsamples and it is sufficiently small so as to be able to assume that, in it, the gas saturation is distributed homogeneously. In such a way, the amount of information that is obtained, which is the sum of that relative to all the subsamples, is thus much greater with respect to that which can be obtained with known methods, in which the rock sample is treated as a single and indivisible object. Moreover, all the criticalities related to the heterogeneity of the distribution of the gas saturation in the sample are overcome by analysing the individual subsamples. The respective Bond number is associated to every subsample and it is possible to determine the critical value of the Bond number from the development of the measured gas saturation. In FIG. 3 the dark points represent the data obtained on seven samples taken from a well; the light points are the results of the measurements carried out on the samples themselves with the known method of Countercurrent imbibition. As can be seen, with the proposed method not only is it possible to obtain many more measurement points, but the trapped gas saturation Sgr in the region of the plateau tends to be smaller and therefore, the estimation is more optimistic. Since the processes of water-gas displacement which are generated in the rock sample better simulate the processes of gas field both in terms of direction of the flow and of fluids used, the method according to the present invention makes it possible to obtain more accurate measurements.

Moreover, the measuring method object of the present invention does not foresee for the laboratory operator to be exposed to harmful substances and, therefore, it is much safer. Indeed, such a method foresees the use of water and it does not have, therefore, any problem of exposure to potentially harmful agents.

Finally, it should be understood that the device thus conceived can undergo numerous modifications and variants, all covered by the invention; moreover, all the details can be replaced by technically equivalent elements. In practice the materials used, as well as the dimensions, can be any according to the technical requirements.

1. A method for measuring trapped gas saturation in a rock sample, comprising:
saturating a porous volume of said rock sample with water;
effecting a first imaging phase capable of dividing the sample into a plurality of subsamples and measuring a water content of each subsample so as to obtain a plurality of porosity values at said plurality of subsamples, said plurality of porosity values forming a porosity map of said sample;
subjecting said sample of water saturated rock to centrifugation in air at a predetermined centrifugation velocity in air;
effecting a second imaging phase so as to obtain a first plurality of gas saturation values Sg at the corresponding subsamples, said first plurality of gas saturation values forming an initial gas saturation map Sgi of said sample;
subjecting said rock sample to centrifugation under water at a pre-determined centrifugation velocity under water;
effecting a third imaging phase so as to obtain a second plurality of gas saturation values at the corresponding subsamples, said second plurality of gas saturation values forming a gas saturation map Sg of said sample;
calculating a plurality of Bond number values corresponding to said centrifugation under water for each of said subsamples;
associating for each subsample, said gas saturation Sg measured with said initial gas saturation Sgi measured and with said Bond number values calculated, generating a Sgi-Sg-Nb tern for each subsample; identifying a Bond number value beyond which the gas saturation Sg begins to decrease, said value identified representing a critical Bond number: and
selecting the initial gas saturation values Sgi and gas saturation values Sg corresponding to Bond number values lower than the value of said critical Bond number, said gas saturation values Sg selected representing corresponding trapped gas saturation values Sgr.

2. The method according to claim 1, wherein said centrifugation phase under water, said third imaging phase, said phase for calculating the Bond numbers Nb, and said association phase, are repeated for a pre-determined number of times.

3. The method according to claim 1, wherein said rock sample is positioned in a cell, said cell comprising a spacer positioned between the base of said cell and said sample, said spacer being arranged to keep said rock sample at a predetermined distance from the bottom of said cell.

4. The method according to claim 1, wherein said imaging phases are effected by a monodimensional or three-dimensional tomographic analysis.

5. The method according to claim 1, wherein said imaging phases are effected by nuclear magnetic resonance (NMR).

6. The method according to claim 1, wherein said imaging phases are effected by Gamma ray analysis.

7. The method according to claim 1, wherein said imaging phases are effected by X-ray analysis.
